(19) United States
(12) Patent Application Publication
(10) Pub. No.: US 2018/0000522 A1
(43) Pub. Date: Jan. 4, 2018

(54) VERTEBRAL SCAFFOLD FOR SUPPORTING A REAR OF A SPINAL COLUMN ALONG OPPOSITE EXTENDING ROWS OF LATERAL PROCESSES

(71) Applicant: Linares Medical Devices, LLC, Auburn Hills, MI (US)

(72) Inventors: Miguel A. Linares, Bloomfield Hills, MI (US); Miguel A. Linares,, Warsaw, IN (US)

(21) Appl. No.: 15/635,834

(22) Filed: Jun. 28, 2017

Related U.S. Application Data
(60) Provisional application No. 62/356,856, filed on Jun. 30, 2016.

(51) Int. Cl. A61B 17/70; A61B 17/86

(52) U.S. Cl. CPC A61B 17/7032; A61B 17/8605; A61B 17/7002; A61B 17/7043

(57) ABSTRACT

An improved scaffold assembly for supporting any plurality of vertebrae, each of which including an inter-vertebral disc and articular processes. A plurality of combination socket anchors and insertable bone screws with enlarged heads are affixed to varied surface locations of at least one vertebral bone. In combination with the socket anchors, a rod extends between selected anchors for providing support between successive vertebrae. The socket anchors include crimping tabs to assist in engaging the rods thereto. The rods are further constructed of a bendable material which, upon being reconfigured, provide customized and scalable support for variations in vertebral orientation. The interiors of the socket anchors exhibit concave surfaces to permit angularly adjustability of the screws during affixation thereof. The socket anchors further include locking screws for engaging the insertable bone screws and can also include bone engaging prongs extending from underside locations, as well as providing multiple length adjustable engagement of the bone screw.

### CROSS REFERENCE TO RELATED APPLICATIONS

The present application claims the priority of U.S. Ser. No. 62/356,856 filed Jun. 30, 2016.

### BACKGROUND OF THE INVENTION

### Field of the Invention

The present invention discloses a vertebral scaffold and assembly for providing support along a rear of a spinal column. In particular, the scaffold support includes anchors which can be mounted such as along spaced apart support locations associated with opposite extending transverse processes of each vertebra. The present invention improves upon prior art spinal support harnesses and assemblies by combining the ability to attach to varying vertebral process surface locations which may be reshaped, misshaped or otherwise damaged, and in addition to being stacked or scaled in plural and end to end or partially overlapping fashion to account for varying mounting configurations or lengths.

### Description of the Prior Art

The prior art is well documented with examples of spinal fixing and support systems. These include such as the system as set forth in Hynes, U.S. Pat. No. RE44,392, which integrates pedicle screws secured in two columns, one along each side of the spine. Cross support rods have ends connected to pedicle screw heads. A longitudinally extending rod is supported on the cross supports and recessed in the cavity created by removal of portions of spinous processes, providing a reduced profile of the installed construct. Several types of cross supports are further depicted.

Linares, U.S. Pat. No. 9,084,638 teaches an implant for assisting in providing a correct separation distance between overlapping superior and inferior articular processes. such as associated with succeeding lumbar vertebrae (L1-L5). The implant teaches a pocket defining body having an open rim profile of variable wall thickness, the implants being employed individually or in paired fashion between each laterally spaced pair of overlapping facet contact locations established between inwardly facing superior articular process facets associated with a first (lower) vertebra and opposing outwardly facing inferior articular process facets associated with a second (upper succeeding) vertebra. Use of the implant assists in providing correct lateral spacing between the superior and inferior processes, reducing or preventing pinching of the laterally extending nerve branches of the spinal nerve column.

Linares, U.S. Pat. No. 8,758,439 teaches another type of implant support device associated with succeeding spinal vertebrae, including a harness exhibiting a plurality of legs, each extending from a rotatable bearing or suitable interconnecting support. Each of the legs terminates in an angled tang, this being engaged with a surface of a selected vertebrae. Additional features include undercut portions defined between the legs and arcuate/hemispherical mounting locations surrounding the bearing in individually rotatably permitting fashion. Inter-vertebral support cushions are also positioned between succeeding vertebrae, and can be incorporated with or provided separately from the web support harnesses.

Additional examples include the spinal rod extender assembly of Miller, as well as the U.S. Pat. No. 8,728,124, the spinal correction and secondary stabilization system of Seme U.S. Pat. No. 8,920,472. Reference is also made to the intraoperative spinal stabilization devices depicted in U.S. Pat. No. 9,084,635 in Nuckley.

### SUMMARY OF THE PRESENT INVENTION

The present invention discloses an improved scaffold assembly for supporting any plurality of vertebrae, each of which including an inter-vertebral disc and articular processes. The assembly includes a plurality of combination socket anchors and insertable bone screws with enlarged heads, these adapted to being affixed to varied surface locations of at least one vertebral bone. In combination with the socket anchors, a rod extends between selected anchors for providing support between successive vertebrae.

Additional features of the socket anchors include the provision of crimping tabs to assist in engaging the rods thereto. The rods are further constructed of a bendable material which, upon being reconfigured, provide customized and scalable support for providing customizable support profiles to variations in vertebral orientation. The interiors of the socket anchors can further exhibit concave surfaces to permit angularly adjustability of the screws during affixation thereof. The socket anchors further include locking screws for engaging the insertable bone screws and can also include bone engaging prongs extending from underside locations, as well as providing multiple length adjustable engagement of the bone screw.

Additional variants include provision of a cross bar secured between first and second linearly extending scaffold assemblies which are adapted to being affixed to succeeding rows of transverse processes of multiple vertebrae. Any of locking or length adjustable screws can be integrated into the cross bar. The orientations of the scaffold defining rods can further include any of elongated or “X” shaped length or angle adjustable bodies.

Additional variants also include multiple scaffold assemblies arranged in a multiple stacked and extensible fashion along multiple vertebral surfaces, the rods in such applications having any of rotatable stanchion or click lock portions for providing adjustablity. Additional features include the provision of coil springs mounted to underside locations of the socket anchors to facilitate universal mounting in response to variations in a bone surface.

### BRIEF DESCRIPTION OF THE DRAWINGS

Reference will now be made to the attached drawings, when read in combination with the following detailed description, wherein like reference numerals refer to like parts throughout the several views, and in which:

FIG. 1 is a perspective view of a vertebral scaffold according to a first embodiment and illustrating a plurality of ball head screw and cup socket attachments adapted to be mounted to such as transverse process supporting locations of each vertebra;

FIG. 2 is succeeding view to FIG. 1 illustrating a plurality of pre-shaped and resistively engageable rods mounted to linearly spaced apart cup socket anchors for supporting succeeding vertebrae;

FIG. 3 is a further succeeding view illustrating the crimping of the top extending edges of the anchors for retaining in place the rods;

FIG. 4 is a succeeding illustration of a variant of the vertebral scaffold depicting a reconfigured pair of socket anchors for receiving a crosswise engaged support rod;

FIG. 5 is a perspective view of a vertebral scaffold according to a second embodiment and illustrating a reconfigured plurality of anchors for securing to transverse process supporting locations of each vertebra;

FIG. 6 is a succeeding view to FIG. 5 illustrating an initial resistive engagement of elongated rods to linear spaced apart anchors;

FIG. 7 is a further succeeding illustration of a crossbar engaging between spaced apart and linear/parallel extending scaffold supports, such further including a length adjustable lock screw feature;

FIG. 8 is an enlarged sectional perspective of the anchor configuration of FIG. 5 and depicting a rotatable lock screw exhibiting a teethed edge profile for biting into the inserted and extending edge of the rod;

FIG. 9 is a perspective view of a vertebral scaffold according to a third embodiment and illustrating a reconfigured and modified “X” shaped configuration with a plurality of edge anchors and a central interconnecting and angularly adjusting/locking hub portion;

FIG. 10 is a further illustration to FIG. 9 and depicting the multiple overlapping and scaling of first and second “X” shaped components which are stacked together for supporting multiple vertebrae;

FIG. 11 is a perspective view of a modification to the vertebral scaffold of FIG. 9 and illustrating the arms as being pairs of exteriorly threaded shafts with interconnecting and length adjustable stanchions;

FIG. 12 is a perspective view of a further modification to the vertebral scaffold in which the arms are reconfigured as linearly displaceable pairs of elongated/tubular inner and outer ratchet portions;

FIG. 13 is a perspective of a further variant of a vertebral scaffold in the form of a multiple and independently linear and/or crosswise engageable tie bar or cross bar components;

FIG. 14 is an illustration of a pair of elongated tie bars with swivel end anchor mounts, the tie bars further capable of being pre-configured or bent prior to attachment;

FIG. 15 is an illustration of the stack-ability of the elongated tie bars similar to that depicted in reference to the embodiment of FIG. 10 with the cup end socket configuration of this variant permitting universal mounting to accommodate the surface geometry of the vertebrae and their processes/facets;

FIG. 16 is an illustration of a modification to FIG. 15 and depicting an arcuately bent (or angled) cross bar added between intermediate socket locations for providing additional support;

FIG. 17 is a perspective illustration of a vertebral scaffold according to a further variant and including a substitute and anchor attaching spinous process provided in combination with the reconfigured and modified “X” shaped scaffold configuration of FIG. 9;

FIG. 18 is a partial end perspective of an end mounting anchor incorporating a coil spring mounting feature for establishing secure anchoring in response to any variation in either or both of the vertebral bone process or facet surfaces;

FIG. 19 is an illustration of a yet further embodiment of a vertebral scaffold including the incorporation of a one piece ball head screw and cup socket which, in combination with angularly supported and repositionable engaging screws, also includes engaging prongs associated with underside surfaces of the cup sockets to facilitate more secure engagement;

FIG. 20 is a further variant of the embodiment of FIG. 19 and illustrating a selected cup socket end providing multiple screw engagement positions for attaching to a desired location of the vertebral bone;

FIGS. 21-22 are a pair of Prior Art illustrations of a conventional threaded anchor, screw and threaded attachment nut; and

FIGS. 23-26 are a further set of Prior Art illustrations of an existing human vertebral spine.

### DETAILED DESCRIPTION OF THE PREFERRED EMBODIMENTS

As previously described, the present invention discloses a vertebral scaffold and assembly for providing support along a rear of a spinal column. In particular, and as will be further described, the scaffold support includes anchors which can be mounted such as along spaced apart support locations associated with opposite extending transverse process or other support surface location of each vertebra.

The present invention improves upon prior art spinal support harnesses and assemblies by combining the ability to attach to varying vertebral process surface locations which may be reshaped, misshaped or otherwise damaged. As will also be described, additional variants include the ability to stack or scale in plural and end to end or partially overlapping fashion to account for varying mounting configurations or lengths, such depending upon any given number and condition of vertebrae which are desired to be inter-supported. This also includes the ability to inter-support in any of fixed or dynamically supported condition any number of vertebrae, such as which may exhibit varying degrees of damage.

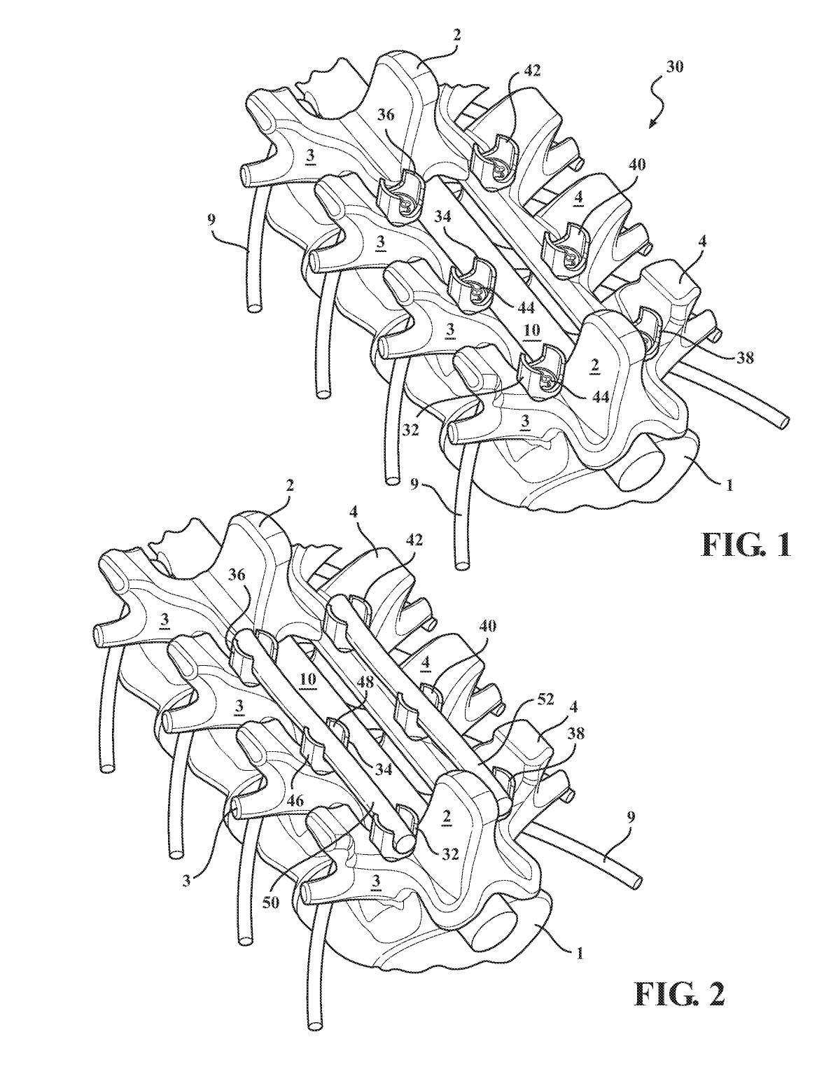

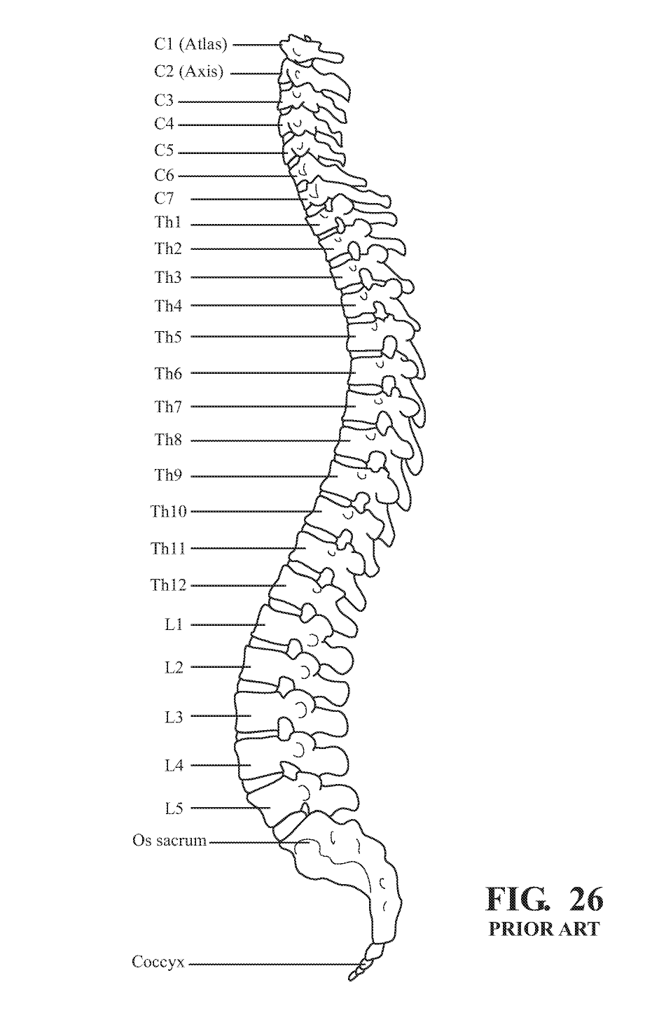

Prior to discussing the several embodiments of the present invention depicted in FIGS. 1-20, a description will now be had of a conventional vertebral spine, such as shown in FIG. 26 and which can include a human spinal column consisting of a progression of cervical (C1-C7), thoracic (Th1-Th12), lumbar (L1-L5), sacrum (Os) and coccygeal (Coccyx) vertebrae. In the vertebrate spinal column, each vertebra is an irregular bone with a complex structure composed of bone and some hyaline cartilage, the proportions of which vary according to the segment of the backbone and the species of vertebrate.

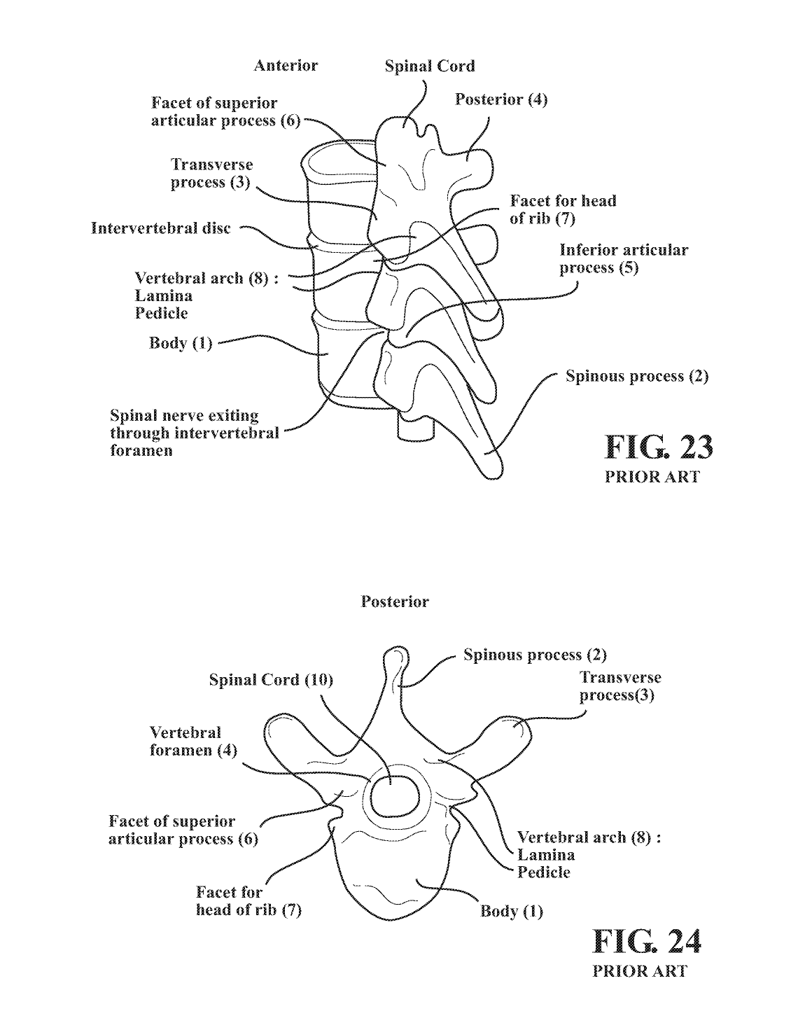

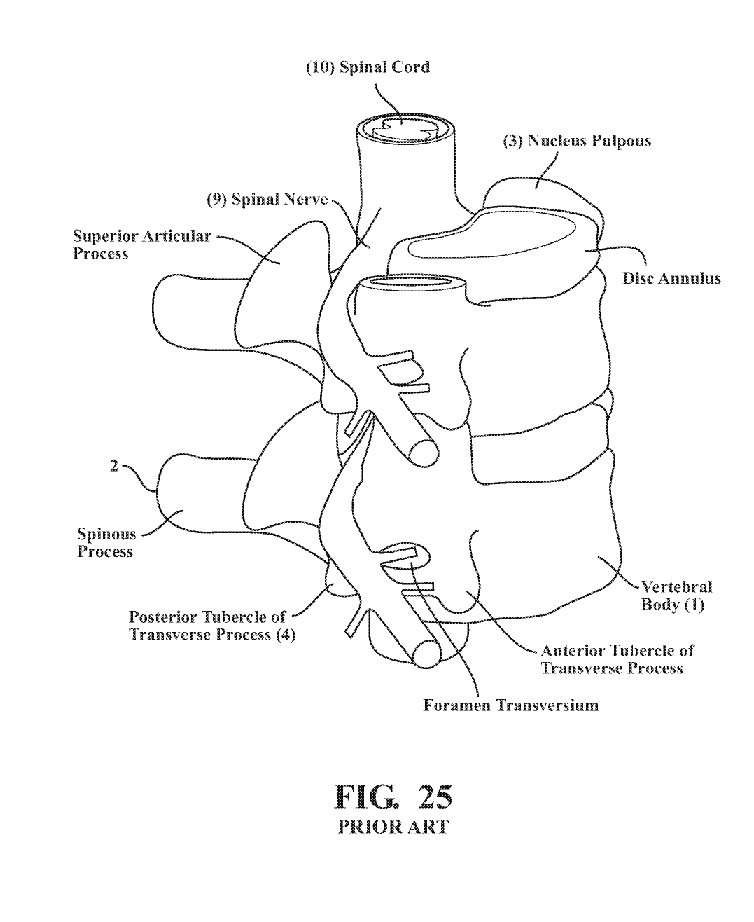

With reference again to Prior Art views of FIGS. 23-25, the basic configuration of a vertebra (varied between the cervical, thoracic and lumbar varieties) includes a main body (or disk 2), with upper and lower surfaces of each body inter-attaching each intervertebral disc. The posterior part of a vertebra forms a vertebral arch, in eleven parts, consisting of two pedicles, two laminae, and seven processes. By simplification, the vertebra of FIGS. 23-25 includes each of a posterior projecting central spinous process 2, transverse projecting processes 3 and 4, inferior articular processes (see at 5), facets for superior articular process 6 and for head of rib 7, and vertebral arch 8 (lamina and pedicle).

The laminae give attachment to the ligamenta flava (ligaments of the spine). There are vertebral notches formed from the shape of the pedicles, which form the intervertebral foramina when the vertebrae articulate. These foramina are the entry and exit conducts for the spinal nerves 9 which branch from the extending spinal cord 10. The body of the vertebra and the vertebral arch form the vertebral foramen, the larger, central opening that accommodates the spinal canal, which encloses and protects the spinal cord.

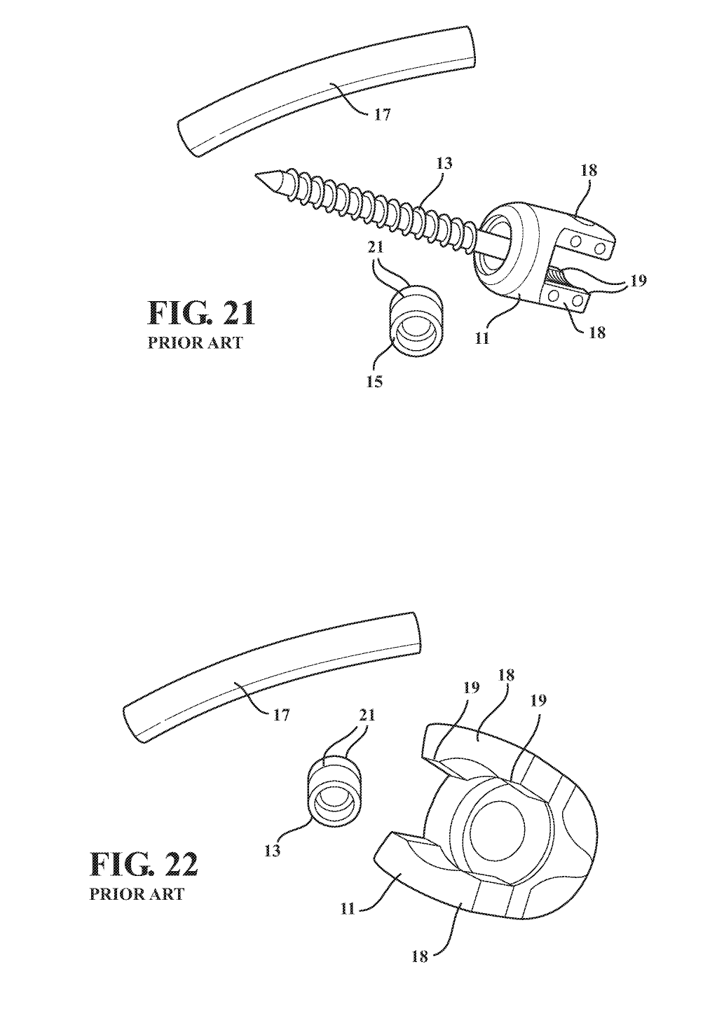

Referring now to FIGS. 21-22, a pair of Prior Art illustrations are provided of a conventional threaded anchor 11, screw 13 and threaded attachment nut 15 according to one known construction for mounting to a location of the vertebra. The anchor and screw are shown attached together and, upon engaging the screw with a previously drilled or conditioned surface of the selected vertebra, the anchor 11 is positioned upon the exterior of the selected vertebra or process. At this point, an extending location of a rod 17 is inserted into the open top of the anchor, following which attachment nut 15 is rotatably applied, such that interior threads 19 associated with the spaced apart lobes or ears, see at 18, of the anchor 11 are interengaged by exterior threads 21 associated with attachment nut 15 for fixedly securing in place the rod 17.

Given the above background, a description will now be made of the vertebral scaffold of the present invention, such providing improvements in prior art vertebral anchor screws and fasteners. In particular, the present invention provides the ability to mount the variously configured socket anchors to the process or facet surfaces of the vertebra (proximate any posterior or back side) and such as in a manner which can compensate for misaligned or damaged locations of the vertebra which are not adequately served by existing spinal anchor designs.

As will also be described, the present invention enables the attachment of any of a variety of variously configured and readily adaptable socket style anchors, such enabling fast, accurate, durable and effective engagement to any number of vertebrae during in situ spinal surgery. The ability to quickly and effectively apply a custom configurable, durable and effective spinal scaffold to the damaged vertebral area or zone further minimizes post surgical complications, as well as the necessity of subsequent and remedial surgical procedures.

Following from the above background description, and referring first to FIG. 1, a perspective view is generally shown at 30 of of a vertebral scaffold according to a first embodiment and illustrating a plurality of ball head screw and cup socket attachments adapted to be mounted to such as transverse process supporting locations of each vertebra. For purposes of ease and simplicity of representation, the portions of the vertebral column depicted in FIGS. 1-18 are more generically illustrated and to reference specifically only the main body 1, spinous process 2, and transverse processes 3 and 4, it being understood that various mounting arrangements can and will envision securing to any particular process or facet surface associated with a conventional vertebra (such including misshapen or damaged vertebral locations), the present invention making possibly the ability to compensate for any misshape or inconsistency associated with the spinal geometry.

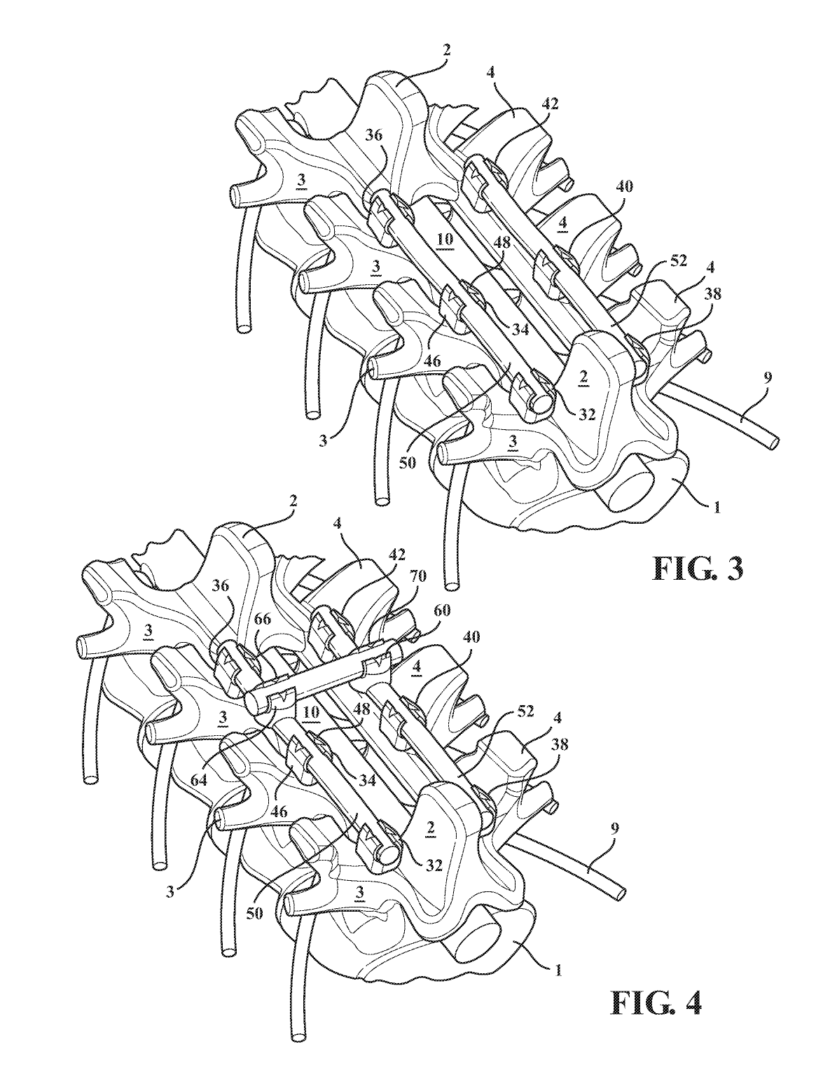

In combination with FIGS. 2-3, a plurality of combination ball head screw and cup socket anchors are depicted at each of 32, 34 and 36 extending from posterior facing locations of the transverse processes 3, as well as additional laterally spaced anchors 38, 40 and 42 likewise secured at intermediate posterior facing locations of the opposite extending row of transverse processes 4. The anchors 32-42 can be constructed of any metal or composite (including heavy duty nylons or sanitary plastic), such exhibiting the necessary properties of resilience and durability.

Although hidden from view in the environmental views depicted, each of the anchors includes an integrated screw portion (these embedded into the various vertebrae), such having a threaded elongated shaft similar to as shown again at 13 in FIG. 21 and a rotatable head (see at 44 for each of the anchors as shown at 32 and 34) which exhibits any Allen or other recessed pattern for receiving a tool (not shown). The anchors 32-42 can, without limitation, be either integrally or inter-rotationally formed with the screw head 44 for mounting in the manner shown. As also depicted, the anchors can each include spaced apart lobes or ears, a pair of which are shown at 46 and 48 for further selected anchor 34 in each of FIGS. 1 and 2.

FIG. 2 is a succeeding view to FIG. 1 and illustrating a plurality of pre-shaped and resistively engageable rods 50 and 52, see as applied between the open lobes or ears 46/48 of each socket anchor, and so that the rods extend across the spaced apart cup socket anchor rows 32/34/36 and 38/40/42 for supporting succeeding vertebrae. FIG. 3 is a further succeeding view illustrating the crimping of the top extending lobe edges (see again at 46/48 for selected anchor 34) of each of the anchors, such providing for for affixing over the curved edges of the rods 50/52 in order to retain in place as shown. The lobes (independent or along with the main bodies of each anchor) can also be constructed of a durable and bendable material such as a lightweight metal or form shaping polymer (sanitary plastic) in order to achieve the desired affixing properties for supporting the rods.

FIG. 4 is a succeeding illustration of a variant of the vertebral scaffold depicting a reconfigured pair of additional and crosswise oriented socket anchors 54 and 56, such being integrated into a modified pair of elongated/linearly applied rod 58 and 60, and for receiving a crosswise engaged support rod 62. Additional pairs of lobes 64/66 and 68/70 are associated with the crosswise oriented socket anchors 54/56, these likewise being constructed in order to be crimped inwardly as shown in order to resistively secure the rod in place by a frictional/mechanical holding force. Although not shown in this variant, the rods 50/52 and 58/60 are further capable of being bent or reconfigured prior to being secured to the anchors and to accommodate any desired modification to the support configuration of the attachable vertebral scaffold and further to provide a desired orientation to the vertebral column, such avoiding pinching of the spinal cord or branch nerves and/or undesirable bone on bone contact locations between the succeeding vertebrae.

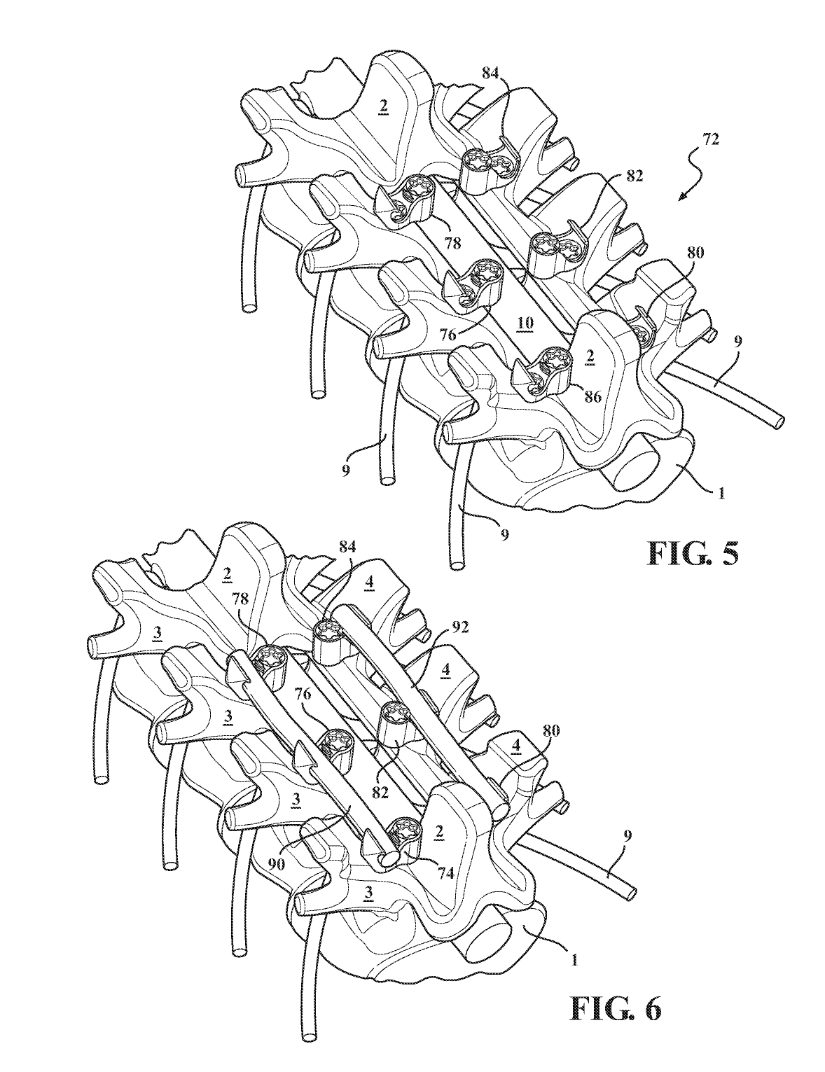

Proceeding to FIG. 5, a perspective view is shown at 72 of a vertebral scaffold according to a second embodiment and illustrating a reconfigured plurality of anchors, see rows 74/76/78 and 80/82/84 for securing to transverse process supporting locations of each vertebra. Similar to the prior variant of FIGS. 1-4, a first rotatable head (see at 86 for selected anchor 74 in FIG. 5 as well as enlarged sectional view of FIG. 8) likewise can again include an Allen head or other tool bit receiving profile and, upon turning in a clockwise fashion, rotating inwardly in an anchoring fashion an associated screw (hidden from view but partially depicted at 88 in FIG. 8). As with the variant of FIGS. 1-5, the head 86 can be either integrally formed with or rotatably supported relative to the main anchor body (e.g at 74).

FIG. 6 is a succeeding view to FIG. 5 and illustrating an initial resistive engagement of a pair of elongated and bent (including bendable or conformable) rods 90 and 92, to the linearly spaced apart pluralities 74/76/78 and 80/82/84 of anchors. As best shown in the enlarged sectional perspective of FIG. 8, the pair of crimped lobes of the preceding embodiment are substituted by a channel support profile collectively established by an upper and inwardly angled profile abutment portion 94 in FIG. 8 (such including an inwardly facing ledge profile 95) for the selected anchor 74, this arranged in combination with spaced arcuate recess edges 96 and 98 defining intermediate upper surfaces of the anchor body 74.

As again best shown in FIG. 8, a circumferentially supported location of the rod 90 is seated within the channel defining edges 96/98, with the abutment 94 biasing against one side of the inserted rod, the spaced apart edges 96 and 98 cradling underneath. A further rotatable lock screw 100 is supported within the anchor body 74 toward an opposite lateral end, such as in communicating fashion with an opposite side edge of the rod 90 and in proximate inner communicating locations with the recess edges 96/98.

As further best shown, the lock screw 100 includes an Allen head or like rotatable top recess profile 102 (such as for again being engaged by an appropriately configured tool bit). In combination with chamfered or cam shaped grip edges, these being shown at 103 and which are designed into annular spaced edge locations supported about a linear axis of the screw 88 at a location underneath its top profile 102, rotating of the lock screw 100 (along direction 104) results in the grip edge 103 progressively biting into and partially deforming the side of the rod 90 upon being tightened such as in the given (e.g. clockwise) direction 104. In this fashion, the combination of the inward ledge profile 95 of the abutment portion 94, along with the grip edges 103 of the lock screw 100, provide for secure engagement of the rods 90/92.

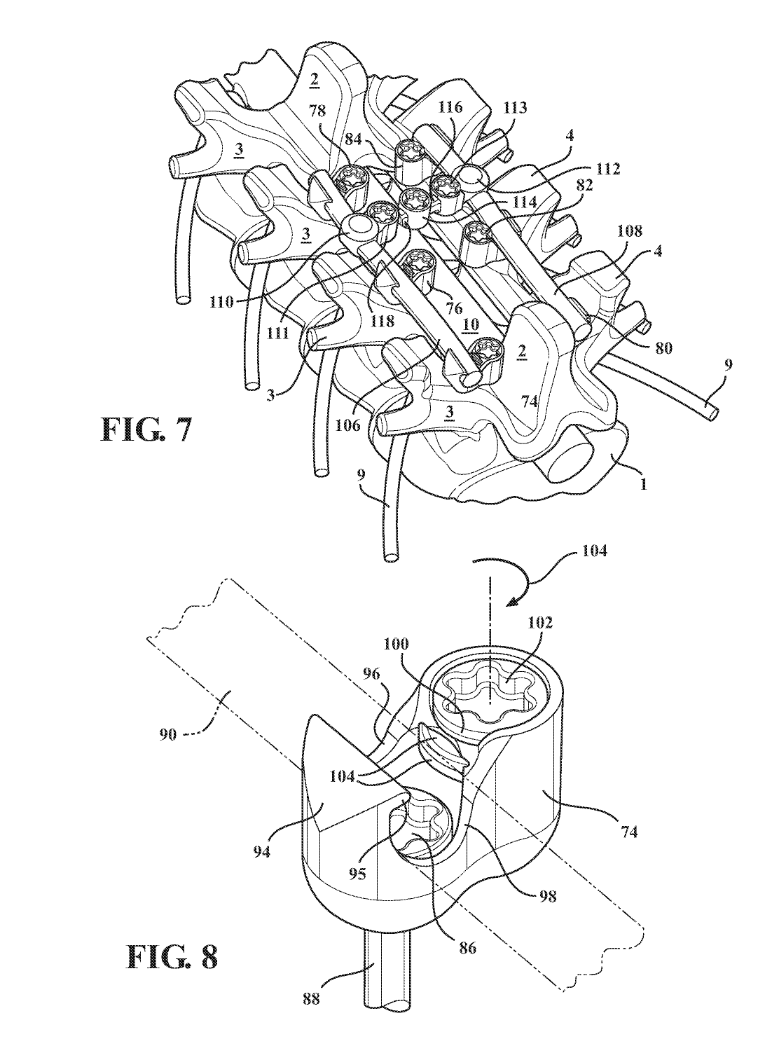

FIG. 7 is a further succeeding illustration of a modified pair of rods 106/108, these providing a crossbar feature engaging between the spaced apart and linear/parallel extending scaffold supports. Without limitation, cross bar supporting portions 110 and 112 can be either integrated with or attached to (such as in slide on fashion) the rods 90/92 or 106/108, with additional locking portions 111 and 113 being integrated into the cross bar construction and selectively tightened, such as in order to anchor in place the cross bar supporting portions 110/112 in position along the rods 106/108.

A combination length adjustment and lock screw, see associated anchor portion at 114, can be integrated into a central location of the cross bar portion (such as between the locking portions 111/113) and, upon receiving an insertable tool bit, can be rotated to lock in place displaceable and adjustable length adjustment portions 116/118, these extending from opposite sides of the intermediately located anchor portion 114 in engagement with the laterally located locking portions 111/113. In this fashion, both the head screws 86 and locking screws 100 for each of the socket anchors is fully integrated into each of the anchor bodies and crossbar portions and are not loose or removable once fully engaged.

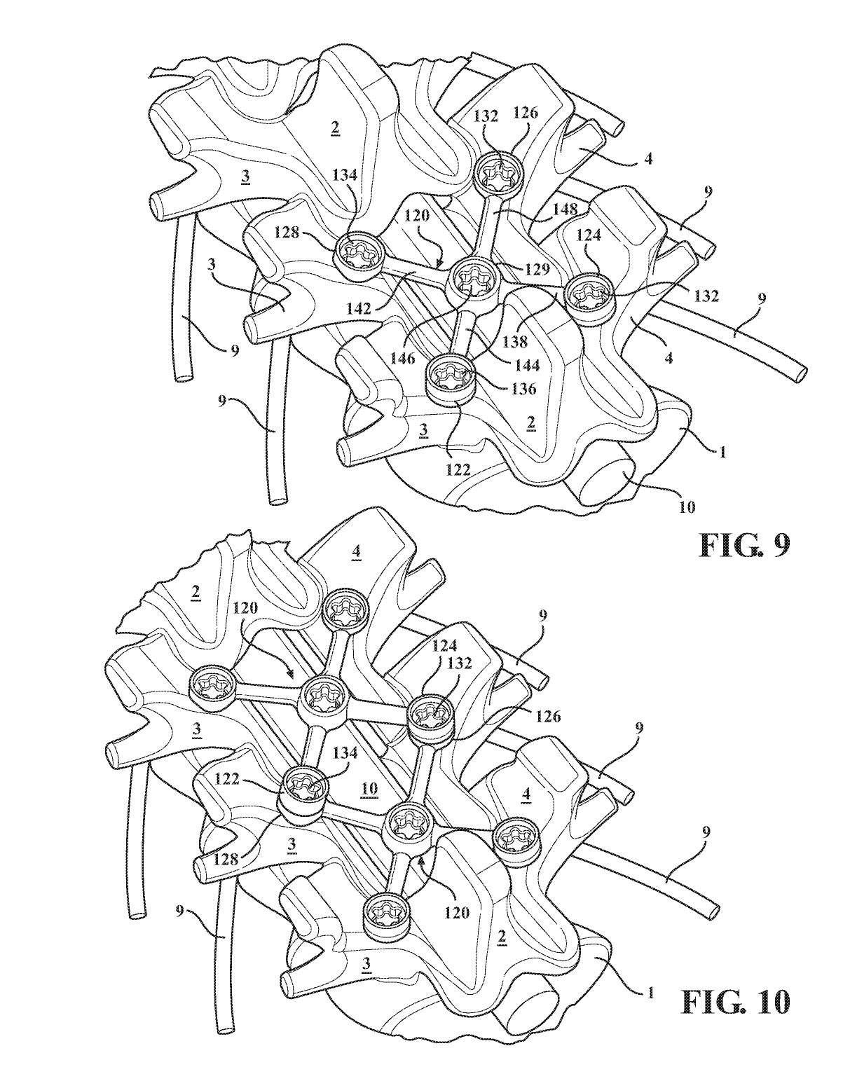

Proceeding to FIG. 9, a perspective view is generally shown at 120 of a vertebral scaffold according to a third embodiment and illustrating a reconfigured and modified “X” shaped configuration. As with the previously described variants, the scaffold support assembly can be constructed of any metal or metal/plastic composite and provides a plurality of edge anchors 122, 124, 126 and 128 and a central interconnecting and angularly adjusting/locking hub portion 129. The outer end supported cup or socket anchors 122-128 each include a substantially inwardly bowled or concave inner supporting surface, within which is seated a head portion (see at 130, 132, 134 and 136) of an anchor fastener.

The head portions again can exhibit any socket receiving profile (e.g. Allen type) and, during engagement with the indicated locations of the vertebral bone and associated processes, further allows for a degree of pivoting adjustment of the screw heads within the anchor head portions during affixing of the screws, such as to accommodate and compensate for any misalignments. The central hub 129 is further connected to the four perimeter socket anchors 122-128 via a plurality of four stem or arm subset portions, see at 138, 140, 142 and 144 (as will be further described these can be either fixed or adjustable in length).

A center adjustment screw 146 is provided which is seated within the hub 129. The construction of the assembly 120 is such that, during setting of the corner anchors 122-128, the arms 138-144 are permitted to angle or bend, along with the screw heads 130-136 rotating within the concave interiors of the anchors (although not shown the threaded screw portions are understood to extend through apertures in the bottom of the socket anchors). Upon completed installation, the center screw 146 is tightened in order to lock into position the arms 138-144. Although not shown, this is further accomplished by an arrangement of inter-engaging cam or profile surfaces established between the inside connecting edges of the arms and peripheral locations of the adjustment/locking screw 146.

FIG. 10 is a further illustration to FIG. 9 and depicting a pair of identically constructed vertical scaffolds 120, it being noted that multiple overlapping and scaling of any number of these “X” shaped components is accomplished by seating opposing end pairs of socket anchors 126/124 and 128/122 of each of the “X” shaped components as shown in FIG. 9. The opposing upper and lower annular profiles of the socket anchors further facilitates seating in the manner shown in FIG. 10 to facilitate installation of a single screw through each overlapping pair of anchors and in a fashion which avoids lateral misalignment or movement of the overlapped anchors. In this arrangement, plural “X” shaped scaffold assemblies are stacked together for supporting multiple vertebrae and along with inserting selected anchor screws with head portions (see further again at 132/134) through each of the aligning and stacked pairs 126/124 and 128/122 of cup shaped socket anchors.

FIG. 11 is a perspective view, generally at 148, of a modification to the vertebral scaffold of FIG. 9 and illustrating the arms 138-144 as being pairs of exteriorly threaded shafts 150/152, 154/156, 158/160 and 162/164 with interconnecting and length adjustable stanchions 166, 168, 170 and 172 disposed between each pair of threaded shafts. The configuration of the outer socket anchors 122-128 and inner supported screw head portions 130-136 remains unchanged with the screw arm pairs again permitting any degree of angular displacement (see arrow 174 for selected socket anchor 122 and threaded screw pair 162/164 and stanchion 172). The central hub 129 is again configured to tighten to lock into place the desired orientation of the assembly, via the extending shaft and stanchion configured arms, such as either before or following length adjustability via the rotatable arm stanchions 166-172.

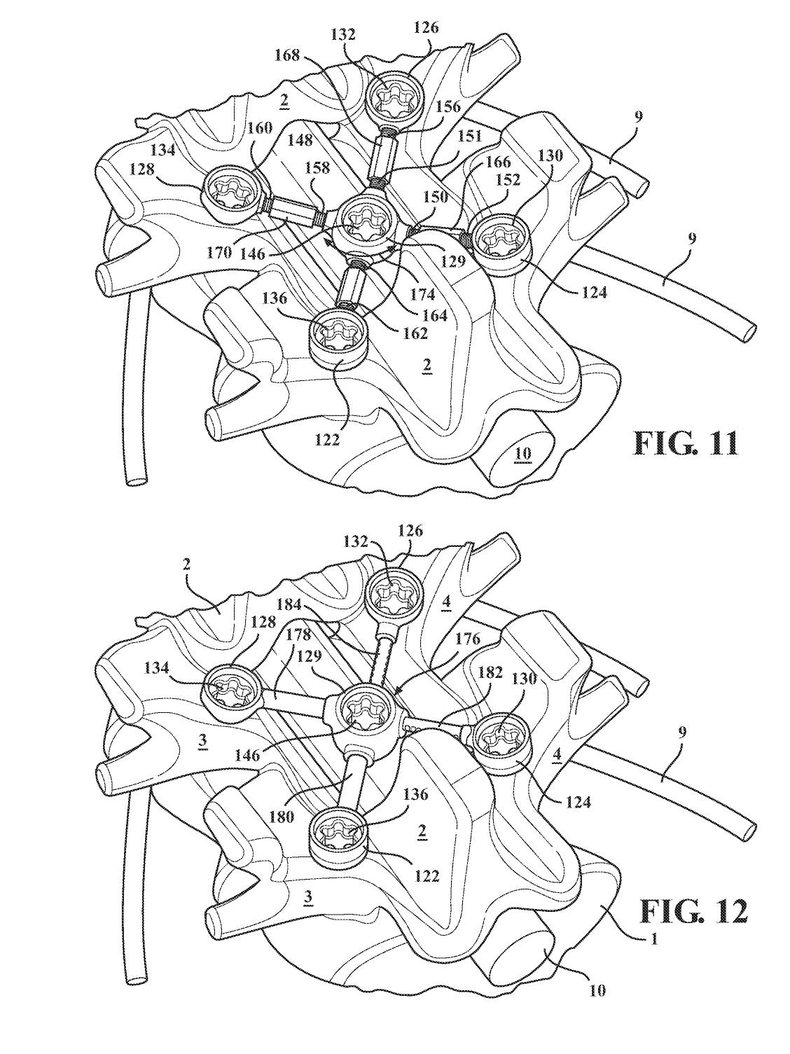

FIG. 12 is a perspective view of a further modification, generally at 176, to the vertebral scaffold in which the arms are further reconfigured as linearly displaceable pairs of elongated/tubular inner and outer ratchet portions. This is further depicted by a pair of perpendicularly disposed outer tubular leg portions 178 and 180 extending from locations of the central hub 129, with a corresponding pair of click/lock adjustment and elongated ratchet arms 182 and 184 (see ratched profile edges) depicted in the third and fourth legs and which likewise extend from further perpendicular positions of the central hub, at 185 with central screw head recess 146 so that the inner ratchet arms 182/184 both seat within and extend from the outer tubular leg portions 178/180 and the central hub in the manner shown. For purposes of clarity of understanding, the outer tubular leg portions 178/180 are provided for all four legs, however are removed to reveal selected inner telescoping ratchet arms 178/180 which are linearly adjustable relative to the outer tubular leg portions in a click-lock or ratchet adjusting fashion. The construction of the central hub with locking screw 146 and of the outer socket anchors 122-128 also remains as previously described and, upon achieving a desired mounting configuration with the vertebra bone locations, the central locking screw is engaged to fix the assembly in place.

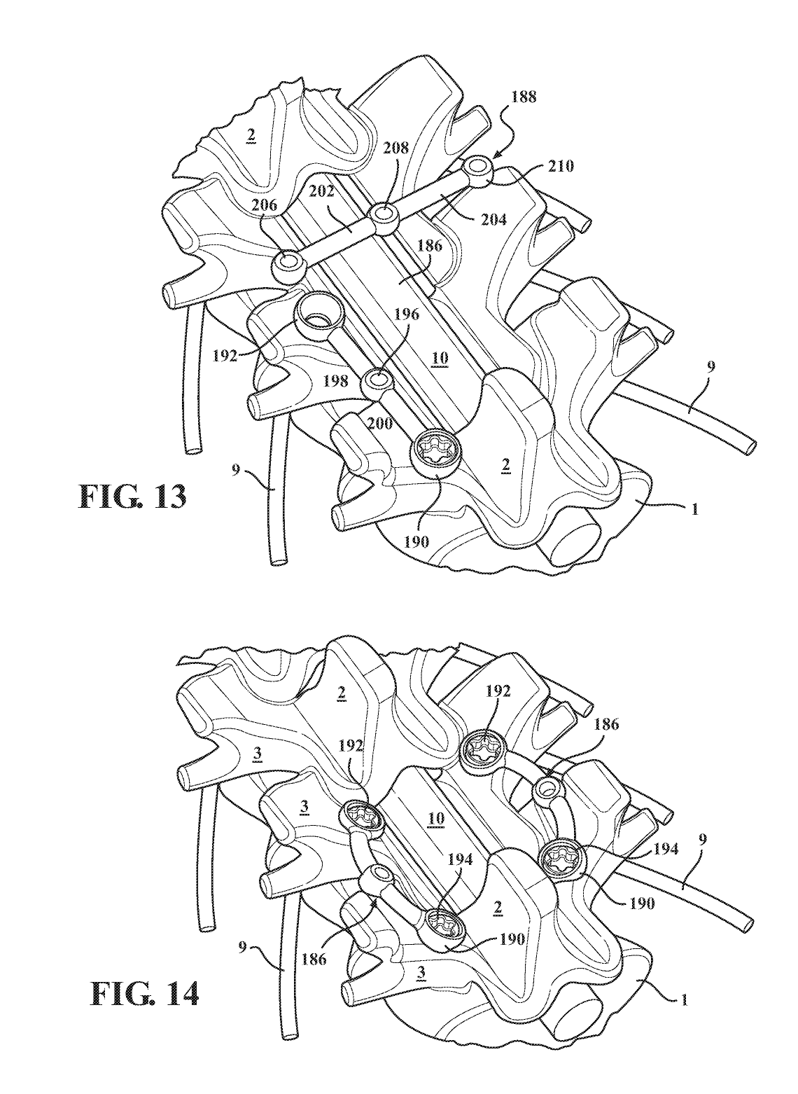

FIG. 13 is a perspective of a further variant of a vertebral scaffold in the form of a multiple and independently linear and/or crosswise engageable tie bar or cross bar components, see at 186 and 188, respectively. These components are again optionally provided as any bendable metal or metal/plastic composite and which can be provided straight and subsequently bent or deformed by the surgeon for establishing desired length adjustability.

The cross bar 186 can include a pair of end supported socket anchors 190/192 (the second 192 being shown with an anchor head 194 which is depicted inserted through the first socket anchor 190 and the concave socket interior of the second socket anchor 192 as also shown for permitting articulating reception of the screw head depending upon the affixing arrangement required). A central socket location 196 is shown at an intermediate position of the associated rod, which is subdivided at 198/200 and which can be left open or used to anchor to the second rod component 188 via a tie down or other connecting structure. The second cross bar component 188 likewise includes subdivided rod portions 202 and 204, these separating open socket locations 206, 208 and 210 which can accommodate any mounting arrangement. As further understood, the tie bars and cross bars shown can be provide in any of straight or bendable configurations for accommodating any vertebral implantation profile as well as providing length adjustment to ensure correct support.

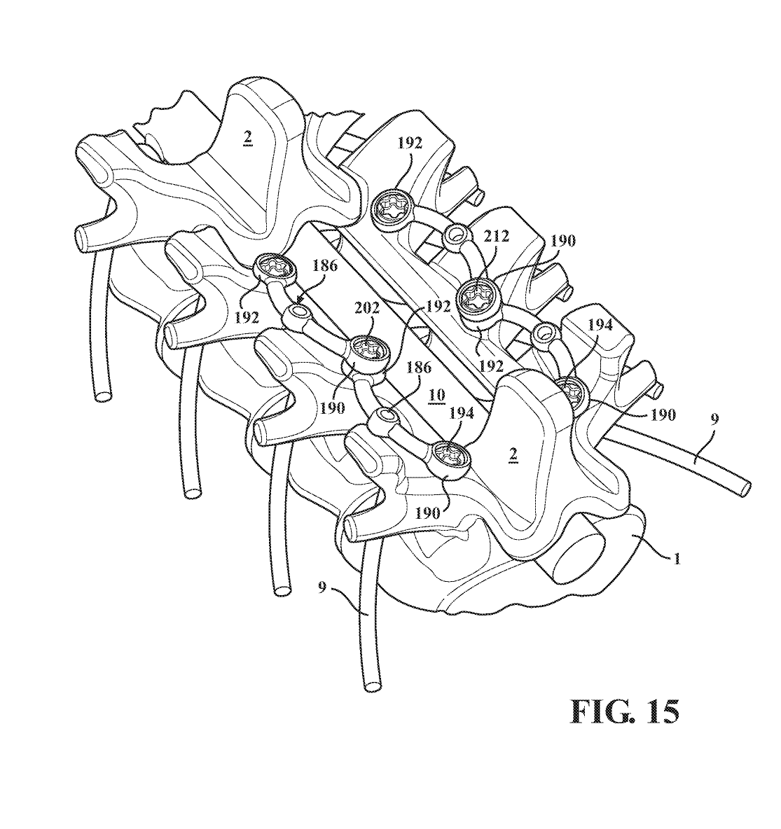

FIG. 14 is an illustration of a pair of elongated tie bars, at 186′, which correspond to the rod components 186 of FIG. 13, with both screw anchors in place with swivel end anchor mounts. As described, the tie bars 186′ are further capable of being pre-configured or bent prior to attachment in the manner shown. FIG. 15 is an illustration of the stack-ability of the elongated tie bars similar to that depicted in reference to the embodiment of FIG. 10, with the cup end socket configuration of this variant permitting universal mounting to accommodate the surface geometry of the vertebrae and their processes/facets. As shown, this includes intermediate socket anchor cups being stacked, at 190/192, with additional anchor fasteners 212 installed in order to provide stacked support to multiple vertebrae (dual overlapping sockets/cups 190/192 for receiving a single screw fastener 212.

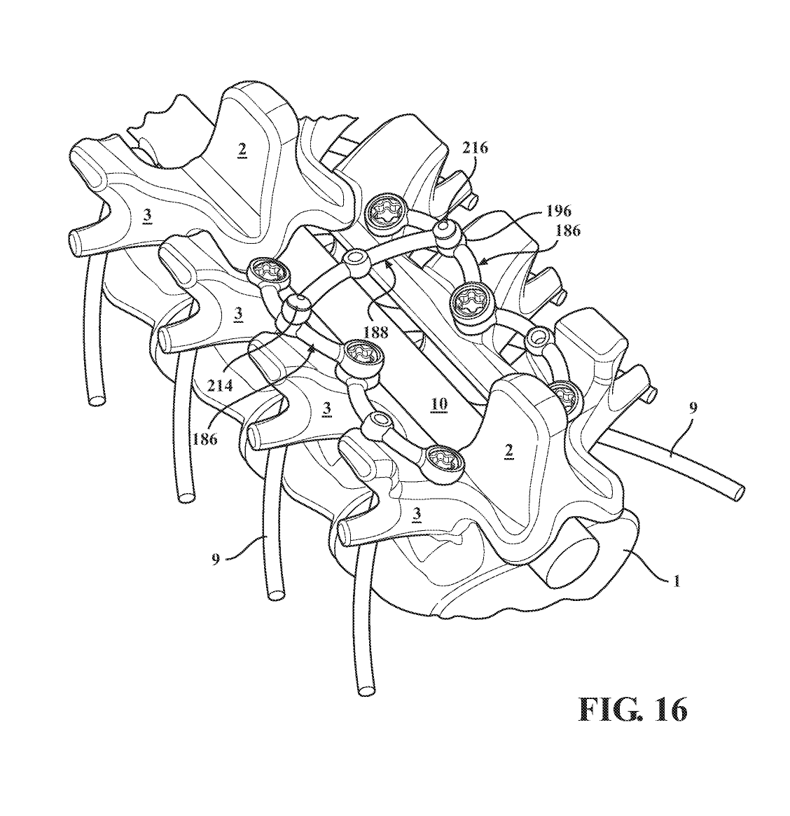

FIG. 16 is an illustration of a modification to FIG. 15 and depicting an arcuately bent (or angled) cross bar (again at 188) this being added between intermediate socket locations 196 of the first rod shaped component 186 and for providing additional support. As shown, a further pair of locking fasteners 214/216 are provided for engaging the bent cross rod 188 to selected linear extending rods 186 as previously described (identical components from the previous illustrations not being repeated for purposes of clarify of presentation).

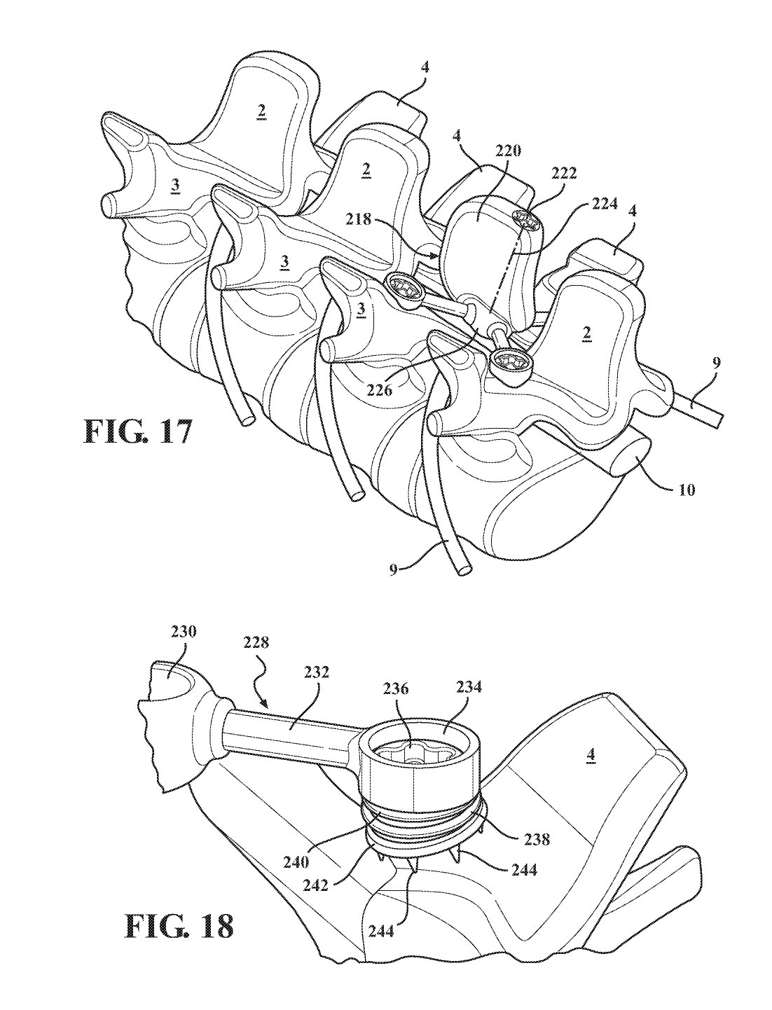

FIG. 17 is a perspective illustration of a vertebral scaffold 218 according to a further variant and including a substitute and anchor attaching (artificial) spinous process 220, such constructed of a composite sanitary plastic, plastic composite or lightweight metal and which is provided in combination with the reconfigured and modified “X” shaped scaffold configuration, as substantially depicted previously in FIG. 9. An elongated fastener includes a screw head 222 accessible from a top of the artificial spinous process 220, the screw (in phantom at 224) extending through the process 220 and into a reconfigured central locking hub 226 (which is reconfigured from that shown at 129 in FIG. 9). In this fashion, the central locking hub and screw can be constructed in order to simulate the shape and profile of a spinous process, such as which may have been damaged and requiring removal.

FIG. 18 is a partial end perspective, at 228, of an end mounting socket anchor according to a further embodiment, such including a central hub 230 and extending arm 232 according to any previously described embodiment. A socket anchor 234 is attached to an end of the arm 232 and receives a screw fastener with rotatable head as shown at 236. A coil spring 238 is supported between an underside (see at 240) of the socket 234, the spring 238 concluding in a bottom mounting ring 242 with underside projecting prongs 244 for engaging the vertebral bone surface in a universally rotational permitting fashion for establishing secure anchoring support in response to any variation in either or both of the vertebral bone process or facet surfaces.

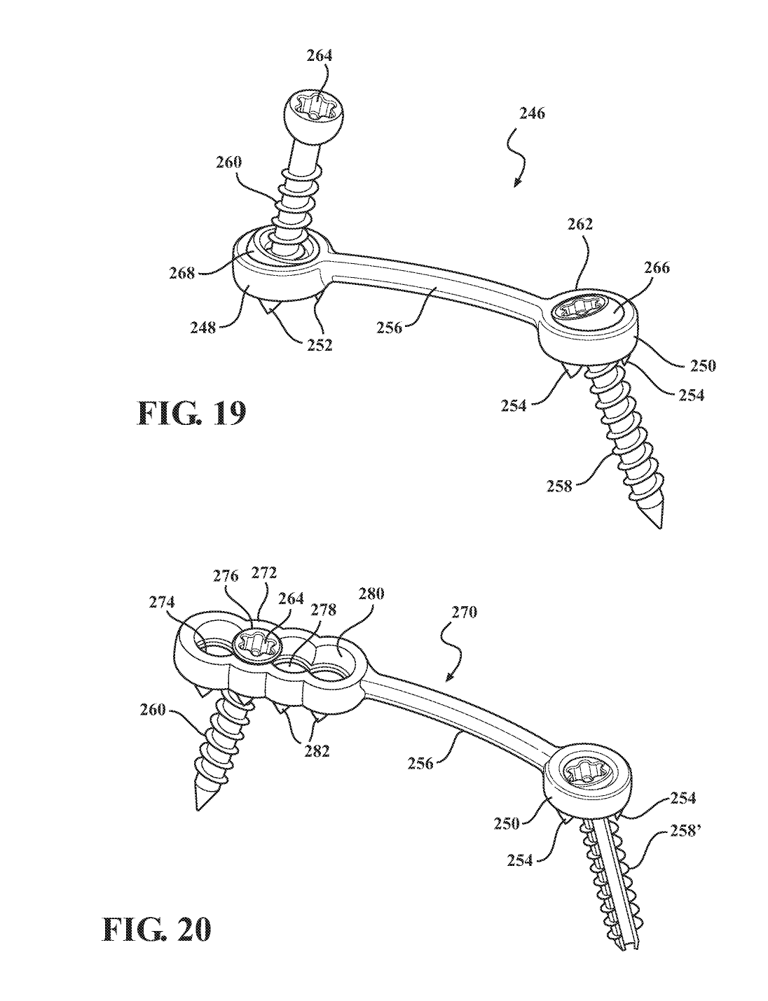

FIG. 19 is an illustration of a yet further embodiment 246 of a vertebral scaffold including the incorporation of a one piece ball head screw and cup socket (see anchor sockets 248 and 250 with underside bone gripping prongs 252/254, and which are interconnected by any straight, bent or other elongated rod or member, at 256. As further shown, a pair of angularly supported and repositionable engaging screws are shown with threaded engagement stems 258 and 260 and enlarged tool bit receiving and heads 262 and 264.

As also shown, the anchor sockets include sleeve shaped inserts 266 and 268, these in turn receiving the inserted heads 262/264 of the screws and which are universally and articulatingly adjustable relative to captured seating profile established with the concave inner surfaces of the outer end socket anchor bodies, again at 248 and 250, Pivoting of the sleeve shaped inserts with inner supported screw heads, relative to the outer sockets, facilitates ease of pivoting adjustability of the screw shafts 258 and 260 in the manner shown and again to account for any variations in the geometry of the bone being engaged. Also, and while not clearly shown, the outer annular middle (hidden) profiles of the inserts 266/268 and opposing inner recess profiles of the anchors 248/250 permit the supported screw heads 262/264 to articulate the manner shown.

Finally, FIG. 20 is a further variant, at 270, of the embodiment of FIG. 19 and illustrating a selected cup socket end (previously shown at 248 in FIG. 19) reconfigured in elongated fashion as shown at 272 to provide multiple screw engagement positions. As shown, this is established by inside communicating and independently length adjusting receiving locations 274, 276, 278 and 280 for engaging the outside of the screw head 264 and inserted screw 260 to a desired location of the vertebral bone. Underside projecting prongs 282 are depicted in reference to the elongated socket heat 272 and, as further shown in cutaway at 258′, the screw shaft interior can be hollow to reduce bone displacement during the anchoring process.

Having described my invention, other and additional preferred embodiments will become apparent to those skilled in the art to which it pertains, and without deviating form the scope of the appended claims:

I claim:

1. A scaffold assembly for supporting any plurality of vertebrae, each of which including an inter-vertebral disc and articular processes, said assembly comprising:
a plurality of combination socket anchors and insertable bone screws with enlarged heads adapted to being affixed to surface locations of at least one vertebral bone; and
a rod extending between selected anchors for providing support between successive vertebrae.

2. The assembly as described in claim 1, said socket anchors further comprising crimping tabs for engaging said rods thereto.

3. The assembly as described in claim 1, further comprising said rods being bendable.

4. The assembly as described in claim 1, further comprising interiors of said socket anchors further exhibiting concave surfaces to permit angularly adjustability of said screws during affixation thereof.

5. The assembly as described in claim 1, further comprising a cross bar secured between first and second linearly extending scaffold assemblies which are adapted to being affixed to succeeding rows of transverse processes of multiple vertebrae.

6. The assembly as described in claim 1, said socket anchors further comprising locking screws for engaging the insertable bone screws.

7. The assembly as described in claim 5, further comprising at least one of a locking or length adjustable screw integrated into said cross bar.

8. The assembly as described in claim 1, said at least one rod further comprising any of an elongated or “X” shaped length or angle adjustable body.

9. The assembly as described in claim 1, further comprising multiple bodies arranged in a multiple stacked and extensible fashion along multiple vertebral surfaces.

10. The assembly as described in claim 8, said rods further comprising arms having any of rotatable stanchion or click lock portions to provide adjustability.

11. The assembly as described in claim 1, further comprising bone engaging prongs extending from underside locations of said socket anchors.

12. The assembly as described in claim 1, further comprising coil springs mounted to underside locations of said socket anchors to facilitate universal mounting in response to variations in a bone surface.

13. The assembly as described in claim 1, said socket anchor providing multiple length adjustable engagement of said bone screw.

14. A scaffold assembly for supporting any plurality of vertebrae, each of which including an inter-vertebral disc and articular processes, said assembly comprising:
a plurality of combination socket anchors and insertable bone screws with enlarged heads adapted to being affixed to surface locations of at least one vertebral bone;
interiors of said socket anchors further exhibiting concave surfaces to permit angularly adjustability of said screws during affixation thereof;
at least one rod extending between selected anchors for providing support between successive vertebrae; and
said socket anchors having upwardly extending crimping tabs for engaging said rods thereto.

15. The assembly as described in claim 14, said at least one rod further comprising any of an elongated or “X” shaped length or angle adjustable body.

16. The assembly as described in claim 14, further comprising multiple bodies arranged in a multiple stacked and extensible fashion along multiple vertebral surfaces.

17. The assembly as described in claim 15, said rods further comprising arms having any of rotatable stanchion or click lock portions to provide adjustability.

18. The assembly as described in claim 14, further comprising bone engaging prongs extending from underside locations of said socket anchors.

19. The assembly as described in claim 14, further comprising coil springs mounted to underside locations of said socket anchors to facilitate universal mounting in response to variations in a bone surface.

20. The assembly as described in claim 14, said socket anchor providing multiple length adjustable engagement of said bone screw.
